SEQUENCE LISTING (2 sequences)

SEQ ID NO: 1 - DNA, 18 bp, Artificial Sequence
  Synthetic oligonucleotide
tctcccagcgtgcgccat

SEQ ID NO: 2 - DNA, 18 bp, Artificial Sequence
  Synthetic oligonucleotide
ucucccagcgtgcgccau

(19) United States
(12) Patent Application Publication
(10) Pub. No.: US 2019/0328763 A1
(43) Pub. Date: Oct. 31, 2019

(54) A LIPID NANOPARTICLE FOR CARRYING ANTISENSE OLIGONUCLEOTIDES INHIBITING BCL-2 AND METHOD FOR PREPARING THE SAME

(71) Applicants: NANJING LUYE PHARMACEUTICAL CO., LTD., Jiangsu (CN); NANJING ASEC NANO BIOMEDICINE CO., LTD., Jiangsu (CN)

(72) Inventors: Guang CHENG, Jiangsu (CN); Wenzhong CHEN, Jiangsu (CN); Lili QIN, Jiangsu (CN)

(21) Appl. No.: 16/342,871

(22) PCT Filed: Oct. 13, 2017
(86) PCT No.: PCT/CN2017/106042

§ 371 Date: Apr. 17, 2019

(30) Foreign Application Priority Data
Oct. 17, 2016 (CN) 201610903392.1

(51) Int. Cl. A61K 31/7088; A61K 9/51; A61K 47/24; A61K 47/26; A61K 47/10; C12N 15/113

(52) U.S. Cl. CPC A61K 31/7088; A61K 9/5123; A61K 9/5192; B82Y 5/00; A61K 47/26; A61K 47/10; C12N 15/113; A61K 47/24

(57) ABSTRACT

A lipid nanoparticle for antisense oligonucleotides inhibiting bcl-2 and belonging to the technical field of biology. The lipid nanoparticle for antisense oligonucleotides inhibiting bcl-2 is prepared by coating an antisense oligonucleotide with a membrane material, and the nucleic acid sequence is 5′-TCT CCC AGC GTG CGC CAT-3′ (SEQ ID NO: 1), or 5′ UCU CCC AGC GTG CGC CAU 3′ (SEQ ID NO: 2). And a method for preparing the same is provided. The nanoparticles have a fairly good inhibitory effect on the growth of tumor cells and specific target genes, and particularly have a fairly good inhibitory effect on KB cervical cancer cells.

### REFERENCE TO SEQUENCE LISTING SUBMITTED VIA EFS-WEB

This application includes an electronically submitted sequence listing in .txt format. The .txt file contains a sequence listing entitled “2019-07-08-Seq.txt” created on Jul. 8, 2019 and is 936 bytes in size. The sequence listing contained in this .txt file is part of the specification and is hereby incorporated by reference herein in its entirety.

### TECHNICAL FIELD

The invention relates to the technical field of biology, in particular to a lipid nanoparticle for antisense oligonucleotides inhibiting bcl-2 and a method for preparing the same.

### BACKGROUND ART

Antisense oligonucleotides generally consist of 18-22 nucleotides and are selectively bound to the target messenger RNA by the principle of complementary base pairing, thereby blocking or inhibiting the function of specific messenger RNAs and regulating the expression of proteins of subsequent target genes.

Bcl-2 is a gene that inhibits apoptosis. This gene can promote cell division, expansion, differentiation, and has higher expression in most tumor cells, thus the tumor can proliferate and metastasize by up-regulating this gene. G3139 is an antisense oligonucleotide consisting of 18 nucleotides having a nucleic acid sequence of 5′-TCT CCC AGC GTG CGC CAT-3′ (SEQ ID NO: 1), which can be bound by the principle of complementary base pairing to messenger RNA encoding the bcl-2, therefore inhibiting the expression of bcl-2 genes and downstream proteins. However, according to previous reports, most antisense oligonucleotides lack a suitable drug delivery system and are difficult to enter cells on their own. They also have a weak binding effect on target messenger RNA and are difficult to maintain the inhibitory binding effect for a long time, and most antisense oligonucleotides are readily degraded under the action of nucleases in the plasma and lose their therapeutic effect. Although G3139 has a certain therapeutic effect after many years of clinical trials, it has finally failed to obtain approval because its therapeutic effect cannot reach the US FDA standard.

There is no prior art where lipid nanoparticle membrane materials include a combination between a Tween compounds and a long-chain polyethylene glycol (PEG) derivative such as TPGS. Tween has been used alone. It has short PEG chains, when can increase the repellency among the nanoparticles to prevent their aggregation and destabilization. On the other hand, due to the lack of long-chain PEG embedded on the surface of nanoparticles, such nano-preparations are easily phagocytosed by phagocytes and lose their function. This is also because Tween is easily lost in the systemic circulation.

Another example is the use of polyethylene glycol (PEG) derivatives alone in nanoparticles, such as TPGS. Although TPGS has a longer PEG chain, it can prevent the recognition by the phagocytic system to a certain extent, and increases systemic circulation time. However, if there are a lot of TPGS, the nanoparticles will be difficult to be effectively taken up by the target tumor cells because of the steric hindrance effect.

For G3139, an antisense oligonucleotide, most of the previous modifications were phosphorothioated (PS) substitutions. Although these modifications can improve the stability of the antisense oligonucleotides to a certain degree, its binding affinity with the messenger RNA was reduced.

### SUMMARY OF THE INVENTION

### 1. Technical Problems to be Solved by the Invention

The purpose of the present invention is to overcome the above technical deficiencies and provide a composition for improving the stability of the nanoparticle itself, thereby promoting the release of the drug in the tumor tissue and reducing the possibility of being degraded, and at the same time improving the resistance of the antisense oligonucleotides to the nucleases and improving the matching accuracy and binding capacity with the messenger RNA.

### 2. Technical Solutions

To achieve the above purpose, the technical solution provided by the present invention is as follows:

a lipid nanoparticle for antisense oligonucleotides inhibiting bcl-2 prepared by coating an antisense oligonucleotide with a membrane material, and the nucleic acid sequence is 5′-TCT CCC AGC GTG CGC CAT-3′ (SEQ ID NO: 1), or 5′ UCU CCC AGC GTG CGC CAU 3′ (SEQ ID NO: 2).

In a further technical solution, the membrane material comprises a cationic lipid, a neutral phospholipid, cholesterol, Tween, a polyethylene glycol derivative in a molar ratio of (25-35):(40-50):(15-25):(1-5):(1-5).

In a further technical solution, the cationic lipid includes: DOTAP, DOTMA, DDAB, and DODMA;

the neutral phospholipid includes: egg PC, DOPC, DSPC, DPPC, and DMPC;

the Tween includes: Tween-20, Tween-40, Tween-60, and Tween-80;

the polyethylene glycol derivative includes: mPEG-DPPE, mPEG-DMPE, mPEG-DSPE, TPGS, and mPEG-cholesterol.

In a further technical solution, the molecular weight of PEG in the polyethylene glycol derivative is from 550 to 5,000.

In a further technical solution, the PEG molecular weights of the polyethylene glycol derivative are 550, 750, 1,000, 2,000, 3,000, and 5,000.

In a further technical solution, the polyethylene glycol (PEG) derivative is a substance formed by linking a hydrophilic polyethylene glycol (PEG) chain or a methylated polyethylene glycol chain (mPEG) to a hydrophobic structure that can be embedded into a phospholipid bilayer; and the hydrophobic structure includes cholesterol, vitamin E, dipalmitoylphosphatidylethanolamine (DPPE), dimyristoylphosphatidylethanolamine (DMPE), dimyristoylglycerol (DMG), or structural analogs.

In a further technical solution, the Tween is Tween-80.

In a further technical solution, a modification to the antisense oligonucleotide 5′ UCU CCC AGC GTG CGC CAU 3′ (SEQ ID NO: 2) is a 2′-O-Me modification to the first three nucleotides at both ends thereof, and a phosphorothioate modification to the entire chain; or phosphorothioate modification to the entire chain of antisense oligonucleotide 5′-TCT CCC AGC GTG CGC CAT-3′ (SEQ ID NO: 1).

A method for preparing a lipid nanoparticle for antisense oligonucleotides inhibiting bcl-2, and the method for preparing the lipid nanoparticle is an ethanol stepwise dilution method comprising the following specific steps:

(1) dissolving a cationic lipid, a neutral phospholipid, cholesterol, Tween, and a polyethylene glycol derivative in 80% ethanol in a certain molar ratio to obtain a mixed ethanol solution; dissolving the antisense oligonucleotides in a PBS buffer to obtain a PBS solution of antisense oligonucleotides;

(2) mixing the resulting mixed ethanol solution and the PBS solution of antisense oligonucleotides in equal volumes to obtain a 40% final ethanol concentration mixed solution;

(3) further diluting the 40% final ethanol concentration mixed solution obtained in step (2) with the PBS solution in an equal volume; repeatedly diluting the final ethanol concentration mixed solution with the PBS solution in an equal volume until a preparation mixed solution with a final ethanol concentration of less than 5% is obtained;

(4) adding a high-salinity solution to the preparation mixed solution obtained in step (3) to obtain a mixed solution containing high-concentration salt;

(5) removing ethanol and free unencapsulated antisense oligonucleotide from the mixed solution obtained in step (4) with an ultrafiltration or dialysis device;

(6) filtering and sterilizing a product obtained in step (5) through a filter membrane or a filter element with a pore size of less than or equal to 0.22 μm to obtain the lipid nanoparticle.

In a further technical solution, the PBS buffer does not contain DNase and RNase, the PBS buffer in the step (1) has a specification of 1× and pH=7, and the PBS buffer in the step (3) has a specification of 1× and pH=7.4.

In a further technical solution, the high-salinity solution is a NaCl solution, and the concentration of NaCl in the mixed solution in step (4) is 0.1-1 M.

According to a further technical solution, the concentration of NaCl in the mixed solution is 0.3-0.4 M, preferably 0.3 M.

In a further technical solution, the ultrafiltration or dialysis device has a molecular weight cutoff of from 10,000 to 100,000 daltons.

In a further technical solution, the membrane material includes DOTAP, Egg PC, cholesterol, Tween 80, and TPGS in a molar ratio of 25:45:20:5:5.

In a further technical solution, the membrane material includes DOTMA, Egg PC, Cholesterol, Tween 80, and TPGS in a molar ratio of 30:45:20:5:5.

In a further technical solution, the membrane material includes DOTAP, DSPC, cholesterol, Tween 80, and TPGS in a molar ratio of 30:50:20:5:5.

In a further technical solution, the membrane material includes DOTAP, Egg PC, cholesterol, Tween 80, and mPEG 2000-DPPE in a molar ratio of 30:45:20:5:5.

DOTAP: 1,2-dioleoyloxy-3-trimethylaminopropyl chloride

DOTMA: 1,2-dioleyloxy-3-trimethylaminopropyl chloride

DDAB: Dioctadecyldimethylammonium bromide

Egg PC: egg yolk phosphatidylcholine

DOPC: dioleoyl phosphatidylcholine

DSPC: distearoyl phosphatidylcholine

DPPC: dipalmitoylphosphatidylcholine

DODMA: 1,2-dioxyoctadecene-3-dimethylaminopropane

DMPC: dimyristoylphosphatidylcholine

DPPE: dipalmitoylphosphatidylethanolamine

DMPE: dimyristoylphosphatidylethanolamine

TPGS: succinate

The phosphorothioate modification is to replace an unbridged oxygen atom in the phosphate group by a sulfur atom. As shown in the figure below, the structure of the entire oligonucleotide chain is less affected, and resistance to various nucleases is improved to a large extent.

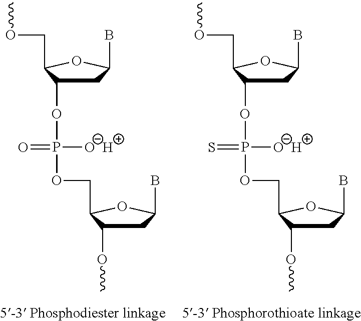

### 3. Beneficial Effects

Compared with the prior art, the present invention has the following significant advantages:

1. For the first time, a dual pegylated reagent (Tween and polyethyleneglycol derivative) was used for the nanoparticle membrane material of the present invention, which can improve the stability of the nanoparticle itself to some extent, and promote release and reduce possibility of degradation of the drug prior to reaching the tumor tissue.

Tween as a pegylated reagent, is usually used in the preparation of nano-preparations. The Tween series have short-chained PEG Tween series such as Tween 20/40/60/80 can improve repulsive interaction among the nanoparticles in nano-preparations, so that the nanoparticles are less prone to aggregation and can improve the stability of nano-preparations to a certain extent, and the particle size growth would be smaller in long-term storage.

The polyethylene glycol derivative such as TPGS can be used as a component of another commonly used nano-preparation, which has a longer PEG chain to promote the circulation of the nanoparticles in the blood and prevent loss due to uptake by reticuloendothelial cells or phagocytes. Recognition of the immune system can be avoided to a certain extent, long circulation time can be achieved, and longer PEG chain will affect the cell uptake to a certain extent.

We found that combination of both, of the longer PEG chain and the shorter Tween, and of these two pegylated reagents by the present invention can effectively improve the stability, long circulation time, target cell release, and gene down regulation efficacy to the target cells of the antisense oligonucleotide-loaded nanoparticles in the plasma.

2. The stability test of the Tween series and the TPGS composition at 4 degrees on the 27th day in the present invention, as shown in FIG. 1, in the one-month time, the change of the particle size of the nano-preparation was smaller, indicating that Tween can maintain the overall stability of the preparation.

The specific experimental data are as follows: in the stability test of Tween 80 at 4 degrees on the 27th day, the nanoparticle size of the nano-preparation was changed from 126 nm to 181 nm, and the stability was most obvious;

in the stability test of Tween 20 at 4 degrees on the 27th day, the nanoparticle size of the nano-preparation was changed from 99.8 nm to 274.2 nm, and the stability was better;

in the stability test of Tween 40 at 4 degrees on the 27th day, the nanoparticle size of the nano-preparation was changed from 138.5 nm to 305.9 nm, and the stability was better;

in the stability test of Tween 60 at 4 degrees on the 27th day, the nanoparticle size of the nano-preparation was changed from 76.7 nm to 218.6 nm, and the stability was better.

3. The membrane material of the lipid nanoparticle of the present invention includes a cationic lipid, a phospholipid, cholesterol, Tween, and a polyethylene glycol derivative which were combined in a certain ratio so that the prepared nano-membrane material can effectively reduce costs, and the stability is good, cell receptivity is high, and it is a good nano-carrier.

4. In the present invention, G3139 was subjected to dual chemical modification of 2′-O-Methyl (2′-O-Me) and phosphorothioate (PS). The first three nucleotides at both ends were subjected to 2′-O-Me modification, which can improve the stability of G3139 oligonucleotide in plasma to a certain extent, while the entire chain was subjected to phosphorothioate modification. Such RNA/DNA/RNA structure can promote RNase H function to specific target messenger RNA sequences for degradation, and improve its resistance to nucleases and improve the matching accuracy and binding ability with messenger RNA to some extent.

When three nucleotides were subjected to 2′-O-Me modification at both ends, the stability of the G3139 antisense oligonucleotide can be greatly improved without affecting the recognition and degradation to the target gene. However, when two (G3139-GAP-LNPs-2) or four nucleotides (G3139-GAP-LNPs-4) were modified by 2′-O-Me, no such effect was produced. The solution provided by the present invention has advantages of good modification effect, being easy to reach the target site, and fairly good inhibitory effect.

5. The nanoparticles of the present invention have a fairly good inhibitory effect on the growth of tumor cells and specific target genes, and particularly have a fairly good inhibitory effect on KB cervical cancer cells.

6. The high-salinity solution added in the preparation of the present invention can dissociate the free oligonucleotides adsorbed on the surface of the nanoparticles, and reduce the larger particle size generated due to the adsorption of the oligonucleotides, and the free oligonucleotides can be removed in a subsequent step of dialysis.

7. The method for preparing the lipid nanoparticle in the present invention is an ethanol stepwise dilution method. However, the method of equivoluminal in-line mixing method used in the prior art is relatively complicated, in which two pumps and different types of liquid storage tanks are required, and the method is not suitable for industrial production. Compared with the prior art, the present invention is more conducive to the dynamic balance of the two systems and promotes the stability of the subsequent suspension system from the perspective of mixing kinetics.

### BRIEF DESCRIPTION OF THE DRAWINGS

FIG. 1 is a graph showing the stability test of the composition of Tween series and TPGS at 4 degrees for 27 days according to the present invention;

FIG. 2 is a graph showing the regulation of gene level (Bcl-2) G3139 in KB cancer cells;

FIG. 3 is a graph showing the regulation of gene level (Bcl-2) G3139 in A549 lung cancer cells;

FIG. 4 is a graph showing the regulation of gene level (Bcl-2) G3139 in LNCaP prostate cancer cells;

FIG. 5 is a graph showing the tumor inhibition rate;

FIG. 6 is a graph showing the survival test.

### DETAILED DESCRIPTION OF THE INVENTION

The present invention and its embodiments are described schematically below, the description is non-limiting, it is only one of the embodiments of the present invention that is shown in the drawings, and the actual structure is not limited thereto. Therefore, if those skilled in the art are inspired by it, structural approach and embodiments similar to the technical solution without creative design should fall within the protection scope of the present invention without departing from the inventive concept of the present invention.

### Example 1

A method for preparing a lipid nanoparticle for antisense oligonucleotides inhibiting bcl-2, comprising the following specific steps:

(1) dissolving DOTAP, Egg PC, cholesterol, Tween 80, and TPGS in a molar ratio of 25:45:20:5:5 in 80% ethanol to obtain an ethanol solution. Dissolve the antisense oligonucleotide 5′-TCT CCC AGC GTG CGC CAT-3′ (SEQ ID NO: 1) in a PBS buffer (1×pH=7) to obtain an antisense oligonucleotide solution;

(2) mixing the resulting mixed ethanol solution and the antisense oligonucleotide solution in equal volumes to obtain a 40% final ethanol concentration mixed solution;

(3) further diluting the 40% final ethanol concentration mixed solution obtained in step (2) with the PBS solution in equal volume; repeatedly diluting the final ethanol concentration mixed solution with the PBS solution (1×pH=7.4) in equal volume until a preparation mixed solution with a final ethanol concentration of less than 5% is obtained;

(4) adding a high-salinity solution to the preparation mixed solution obtained in step (3) to obtain a 0.1 M mixed solution to dissociate the free oligonucleotide bound to the surface of the nanoparticles.

(5) removing ethanol and free antisense oligonucleotides from the mixed solution obtained in step (4) by an ultrafiltration device having a molecular weight cutoff of 10,000 daltons.

(6) filtering and sterilizing a product obtained in step (5) through a filter membrane or a filter element with a pore size of 0.22 μm to obtain the lipid nanoparticle.

### Example 2

A method for preparing a lipid nanoparticle for antisense oligonucleotides inhibiting bcl-2, comprising the following specific steps:

(1) dissolving DOTMA, DOPC, Cholesterol, Tween 40, and mPEG2000-DPPE in a molar ratio of 35:40:15:1:1 in 80% ethanol to obtain an ethanol solution. Dissolve the antisense oligonucleotide 5′UCU CCC AGC GTG CGC CAU 3′ (SEQ ID NO: 2) with phosphorothioate modification on the entire chain, modifying both ends by 2′-O-Me and dissolving in a PBS buffer (1×pH=7) to obtain an antisense oligonucleotide solution;

(2) mixing the resulting mixed ethanol solution and the antisense oligonucleotide solution in equal volume to obtain a 40% final ethanol concentration mixed solution;

(3) further diluting the 40% final ethanol concentration mixed solution obtained in step (2) with the PBS solution in equal volume; repeatedly diluting the final ethanol concentration mixed solution with the PBS solution (1×pH=7.4) in equal volume until a preparation mixed solution with a final ethanol concentration of less than 5% is obtained;

(4) adding a high-salinity solution to the preparation mixed solution obtained in step (3) to obtain a mixed solution having a final concentration of 0.3 M and containing high-concentration salt;

(5) removing ethanol and free antisense oligonucleotides from the mixed solution obtained in step (4) by an ultrafiltration device having a molecular weight cutoff of 50,000 daltons;

(6) filtering and sterilizing a product obtained in step (5) through a filter membrane or a filter element with a pore size of 0.2 μm to obtain the lipid nanoparticle.

### Example 3

A method for preparing a lipid nanoparticle for antisense oligonucleotides inhibiting bcl-2, comprising the following specific steps:

(1) dissolving DDAB, DSPC, cholesterol, Tween 60, and mPEG2000-DPPE in a molar ratio of 30:50:25:3:3 in 80% ethanol to obtain an ethanol solution. Dissolve the antisense oligonucleotide 5′UCU CCC AGC GTG CGC CAU 3′ (SEQ ID NO: 2) with phosphorothioate modification on the entire chain, and modifications on both ends by 2′-O-Me, in a PBS buffer (1×pH=7) to obtain an antisense oligonucleotide solution;

(2) mixing the resulting mixed ethanol solution and the antisense oligonucleotide solution in equal volume to obtain a 40% final ethanol concentration mixed solution;

(3) further diluting the 40% final ethanol concentration mixed solution obtained in step (2) with the PBS solution in equal volume; repeatedly diluting the final ethanol concentration mixed solution with the PBS solution (1×pH=7.4) in equal volume until a preparation mixed solution with a final ethanol concentration of less than 5% is obtained;

(4) adding a high-salinity solution to the preparation mixed solution obtained in step (3) to obtain a mixed solution having a final concentration of 1 M and containing high-concentration salt;

(5) removing ethanol and free antisense oligonucleotides from the mixed solution obtained in step (4) by an ultrafiltration device having a molecular weight cutoff of 100,000 daltons;

(6) filtering and sterilizing a product obtained in step (5) through a filter membrane or a filter element with a pore size of 0.22 μm to obtain the lipid nanoparticle.

### Example 4

| | Particle size | Potential | Encapsulation rate |
|---|---|---|---|
| Preparation | (nm) | (mV) | (%) |
| 1 | 123.4 | 7.45 | 89.2% |
| 2 | 132.5 | 7.96 | 83.8% |
| 3 | 128.1 | 8.55 | 84.9% |

1 DOTAP/Egg PC/Cholesterol/Tween 80/TPGS = 25:50:20:5:5
2 DOTAP/Egg PC/Cholesterol/Tween 80/TPGS = 30:50:20:5:5
3 DOTAP/Egg PC/Cholesterol/Tween 80/TPGS = 35:50:20:5:5

A single-factor experiment was performed by adjusting the molar ratio of DOTAP-positive phospholipids, we found that under the condition that other components were always the same, the particle size and encapsulation rate of the first group were significantly superior than those of the other two groups, the potential remained substantially the same, and therefore we selected the first group as the optimal prescription for the following screening.

| | Particle size | Potential | Encapsulation rate |
|---|---|---|---|
| Preparation | (nm) | (mV) | (%) |
| 1 | 138.6 | 7.53 | 83.6% |
| 2 | 123.4 | 7.45 | 89.2% |
| 3 | 136.0 | 7.21 | 84.2% |

1 DOTAP/Egg PC/Cholesterol/Tween 80/TPGS = 25:40:20:5:5
2 DOTAP/Egg PC/Cholesterol/Tween 80/TPGS = 25:45:20:5:5
3 DOTAP/Egg PC/Cholesterol/Tween 80/TPGS = 25:50:20:5:5

A single-factor experiment was performed by adjusting the molar ratio of Egg PC phospholipids, we found that under the condition that other components were always the same, the particle size and encapsulation rate of the second group were significantly superior than those of the other two groups, the potential remained substantially the same, and therefore we selected the second group as the optimal prescription for the following screening.

| | Particle size | Potential | Encapsulation rate |
|---|---|---|---|
| Preparation | (nm) | (mV) | (%) |
| 1 | 120.4 | 7.78 | 80.4% |
| 2 | 123.4 | 7.45 | 89.2% |
| 3 | 143.4 | 7.03 | 86.2% |

1 DOTAP/Egg PC/Cholesterol/Tween 80/TPGS = 25:45:15:5:5
2 DOTAP/Egg PC/Cholesterol/Tween 80/TPGS = 25:45:20:5:5
3 DOTAP/Egg PC/Cholesterol/Tween 80/TPGS = 25:45:25:5:5

A single-factor experiment was performed by adjusting the molar ratio of cholesterol, we found that under the condition that other components were always the same, the particle size and encapsulation rate of the second group were significantly superior than those of the other two groups, the potential remained substantially the same, and therefore we selected the second group as the optimal prescription for the following screening.

| | Particle size | Potential | Encapsulation rate |
|---|---|---|---|
| Preparation | (nm) | (mV) | (%) |
| 1 | 135.4 | 9.23 | 78.4% |
| 2 | 128.0 | 8.53 | 84.7% |
| 3 | 123.4 | 7.45 | 89.2% |

1 DOTAP/Egg PC/Cholesterol/Tween 80/TPGS = 25:45:20:1:5
2 DOTAP/Egg PC/Cholesterol/Tween 80/TPGS = 25:45:20:3:5
3 DOTAP/Egg PC/Cholesterol/Tween 80/TPGS = 25:45:20:5:5

A single-factor experiment was performed by adjusting the molar ratio of Tween 80, we found that under the condition that other components were always the same, the particle size and encapsulation rate of the third group were significantly superior than those of the other two groups, the potential remained substantially the same, and therefore we selected the third group as the optimal prescription for the following screening.

| | Particle size | Potential | Encapsulation rate |
|---|---|---|---|
| Preparation | (nm) | (mV) | (%) |
| 1 | 129.2 | 8.53 | 82.6% |
| 2 | 124.6 | 7.91 | 79.3% |
| 3 | 123.4 | 7.45 | 89.2% |

1 DOTAP/Egg PC/Cholesterol/Tween 80/TPGS = 25:45:20:5:1
2 DOTAP/Egg PC/Cholesterol/Tween 80/TPGS = 25:45:20:5:3
3 DOTAP/Egg PC/Cholesterol/Tween 80/TPGS = 25:45:20:5:5

A single-factor experiment was performed by adjusting the molar ratio of TPGS, we found that under the condition that other components were always the same, the particle size and encapsulation rate of the third group were significantly superior than those of the other two groups, the potential remained substantially the same, and therefore we selected the third group as the optimal prescription for the following screening.

| | Particle size | Potential | Encapsulation rate |
|---|---|---|---|
| Preparation | (nm) | (mV) | (%) |
| 1 | 131.4 | 5.76 | 88.4% |
| 2 | 127.2 | 6.66 | 91.9% |
| 3 | 126.4 | 6.75 | 84.6% |

1 DOTMA/Egg PC/Cholesterol/Tween 80/TPGS = 25:45:20:5:5
2 DOTMA/Egg PC/Cholesterol/Tween 80/TPGS = 30:45:20:5:5
3 DOTMA/Egg PC/Cholesterol/Tween 80/TPGS = 35:45:20:5:5

In addition, we also screened for other potential phospholipids, a single-factor experiment was performed by adjusting the molar ratio of DOTMA positive phospholipids, we found that under the condition that other components were always the same, the particle size and encapsulation rate of the second group were significantly superior than those of the other two groups, the potential remained substantially the same, and therefore we selected the second group as the optimal prescription for the following screening.

| | Particle size | Potential | Encapsulation rate |
|---|---|---|---|
| Preparation | (nm) | (mV) | (%) |
| 1 | 124.4 | 7.12 | 82.8% |
| 2 | 126.0 | 8.04 | 87.1% |
| 3 | 131.8 | 7.71 | 89.9% |

1 DOTAP/DSPC/Cholesterol/Tween 80/TPGS = 30:40:20:5:5
2 DOTAP/DSPC/Cholesterol/Tween 80/TPGS = 30:45:20:5:5
3 DOTAP/DSPC/Cholesterol/Tween 80/TPGS = 30:50:20:5:5

A single-factor experiment was performed by adjusting the molar ratio of DSPC phospholipids, we found that under the condition that other components were always the same, the particle size and encapsulation rate of the third group were significantly superior than those of the other two groups, the potential remained substantially the same, and therefore we selected the third group as the optimal prescription for the following screening.

| | Particle size | Potential | Encapsulation rate |
|---|---|---|---|
| Preparation | (nm) | (mV) | (%) |
| 1 | 142.6 | 7.68 | 85.2% |
| 2 | 134.5 | 6.63 | 87.3% |
| 3 | 135.8 | 7.90 | 91.4% |

1 DOTAP/Egg PC/Cholesterol/Tween 80/DPPE-mPEG2000 = 30:45:20:5:1
2 DOTAP/Egg PC/Cholesterol/Tween 80/DPPE-mPEG2000 = 30:45:20:5:3
3 DOTAP/Egg PC/Cholesterol/Tween 80/DPPE-mPEG2000 = 30:45:20:5:5

A single-factor experiment was performed by adjusting the molar ratio of DPPE-mPEG2000, we found that under the condition that other components were always the same, the particle size and encapsulation rate of the third group were significantly superior than those of the other two groups, the potential remained substantially the same, and therefore we selected the third group as the optimal prescription for the following screening.

After the composition of the above prescriptions and the screening of proportions, we can conclude that (1) DOTAP/Egg PC/Cholesterol/Tween 80/TPGS=25:45:20:5:5, the composition and ratio have smaller and stable particle size and encapsulation rate, and the positive potential is also relatively suitable. In addition, DOTMA/Egg PC/Cholesterol/Tween 80/TPGS=30:45:20:5:5, DOTAP/DSPC/Cholesterol/Tween 80/TPGS=30:50:20:5:5, DOTAP/Egg PC/Cholesterol/Tween 80/-mPEG 2000-DPPE=30:45:20:5:5 are also preferred ratios among particle sizes, potentials, encapsulation rates, and stability in other component compositions. We will conduct further analysis experiments with these ratios and prescription compositions.

The following experiments were conducted for Example 1: free 2′-O-Me modified G3139 and 2′-O-Me modified G3139 lipid nanoparticles in the table represent G3139 (5′-UCU CCC AGC GTG CGC CAU-3′) (SEQ ID NO: 2) conducting phosphorothioate modification to the entire chain and 2′-O-Me modification to the 3 nucleotides on each end;

Free G3139 and G3139 lipid nanoparticles represent G3139 (5′-TCT CCC AGC GTG CGC CAT-3′) (SEQ ID NO: 1) conducting phosphorothioate modification to the entire chain;

The blank nanoparticles indicate that the non-content lipid nanoparticles were encapsulated by the membrane material.

1. A549 Lung Cancer Cytotoxicity Test

(1) We placed A549 cells in 96-well plates, and made the cells grow in an RPMI1640 culture solution containing 10% fetal calf serum to allow the cells to grow overnight to achieve a plating rate of 60-70%.

(2) A toxicity test of multiple paclitaxel (PTX) concentration gradients (1 nmol/L-100 μmol/L) and antisense oligonucleotide concentrations (10 μmol/L) in each group was conducted with each concentration group on the second day, and each concentration group was incubated in a 37 degree and 5% CO2 environment.

(3) 20 ul of MTS reagent (a novel tetrazole compound) was added at different time points 24 h, 48 h, and 72 h, and the detection was performed at a wavelength of 490 nm and the results were as shown in the following table:

| | IC50 | IC50 | IC50 |
|---|---|---|---|
| Therapeutic | (nmol/L) | (nmol/L) | (nmol/L) |
| effect groups | after 24 h | after 48 h | after 72 h |
| Paclitaxel (PTX) alone | 75.6 | 28.5 | 17.85 |
| PTX + free | 68.4 | 19.4 | 10.73 |
| 2′-O-Me modified | | | |
| G3139 | | | |
| PTX + free G3139 | 65.6 | 17.3 | 13.5 |
| PTX + blank | 88.4 | 31.5 | 15.9 |
| nanoparticles | | | |
| PTX + G3139 lipid | 57.6 | 11.5** | 9.9 |
| nanoparticles | | | |
| PTX + 2′-O-Me | 73.5 | 8.53*** | 7.11** |
| modified G3139 | | | |
| lipid nanoparticles | | | |

**represents p < 0.01
***< represents p < 0.001

In this experiment, each experimental group was treated with different concentrations of paclitaxel (PTX), and the concentration of different antisense oligonucleotide groups was fixed to be 10 umol/L to show its chemical sensitization. We found that 48 h after the treatment of cancer cells, G3139 lipid nanoparticles and 2′-O-Me modified G3139 lipid nanoparticles can have chemical sensitization effect on the cancer cells to a certain extent, if it is compatible with paclitaxel for the treatment, the killing effect on cancer cells can be improved to a large extent, and there was statistically significant.

2. Gene Level (Bcl-2) G3139 Regulation Experiment

By designing different experimental sample groups, we determined the regulatory effect of various G3139 dosage forms on the bcl-2 levels of a series of different cancer cells (including A549 cancer cells, KB cancer cells, and LNCap prostate cancer cells), and β-actin as an internal reference control gene, does not change with the drug treatment, bcl-2 is our target gene, and whether the drug works can be determined by observing its up and down regulations.

(1) We will place cancer cells, such as A549 cells, KB cells, and LNCaP prostate cancer cells in 6-well plates and make the cells grow in an RPMI1640 culture solution containing 10% fetal calf serum to allow the cells to grow overnight to achieve a plating rate of 80%.

(2) G3139 concentration preparation having 1 μM of final concentration was added to a tumor cell culture solution, and the preparation was divided into the following six groups, namely 2′-O-Me modified G3139 lipid nanoparticles, G3139 lipid nanoparticles, free G3139, free 2′-O-Me modified G3139, blank lipid nanoparticles, and culture solution control group.

(3) After incubation in an environment of 37 degrees and 5% CO2 for 4 h, the cells will be transferred to a fresh culture solution and the culture will last for 48 h. Cells were collected, lysed, and extracted RNA according to standard methods.

(4) We will analyze the levels of bcl-2 messenger RNA in each group with the Real-Time-PCR technology, and we can finally quantify the expression of bcl-2 after treatment in each group according to the standard methods of reagent suppliers.

1) KB Cancer Cells

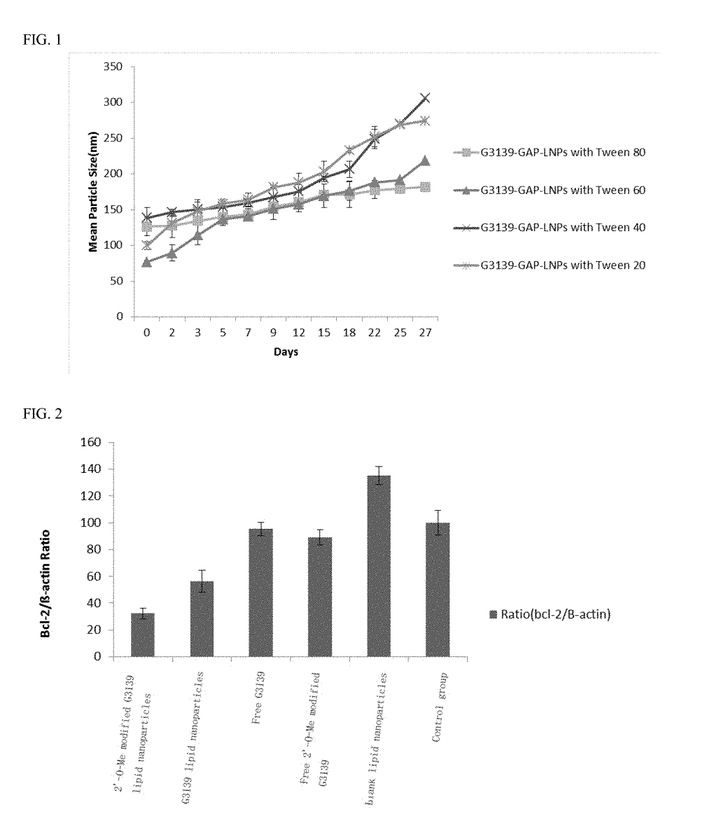

By designing different sample groups, we regulate the levels of bcl-2 in KB cells. The test results are as shown in the following table and FIG. 2, β-actin is an internal reference control gene and does not change with the treatment, bcl-2 is our target gene, and whether the drug works can be determined by target gene regulations.

| Sequence | Therapeutic | Ratio | Standard |
|---|---|---|---|
| numbers | effect groups | (bcl-2/β-actin) | deviation |
| 1 | 2′-O-Me modified G3139 lipid | 32.29% | 4.14% |
| | nanoparticles | | |
| 2 | G3139 lipid nanoparticles | 56.28% | 8.08% |
| 3 | free G3139 | 95.31% | 5.11% |
| 4 | free 2′-O-Me modified G3139 | 89.04% | 5.66% |
| 5 | blank lipid nanoparticles | 135.28% | 9.11% |
| 6 | control group | 100.00% | 4.10% |

100.00% 4.10% From the above table and FIG. 2, the 2′-O-Me modified lipid nanoparticles have a 67.71% gene-level down-regulation, as compared with other experimental groups, the bcl-2 level has a large degree of down-regulation, while the free drug group (3,4) cannot enter the cells due to the lack of a drug delivery system, but only has a weak downregulation (4.69% and 10.96%), and blank nanoparticles cannot play a role of down-regulation because there is no drug.

2) A549 Lung Cancer Cells

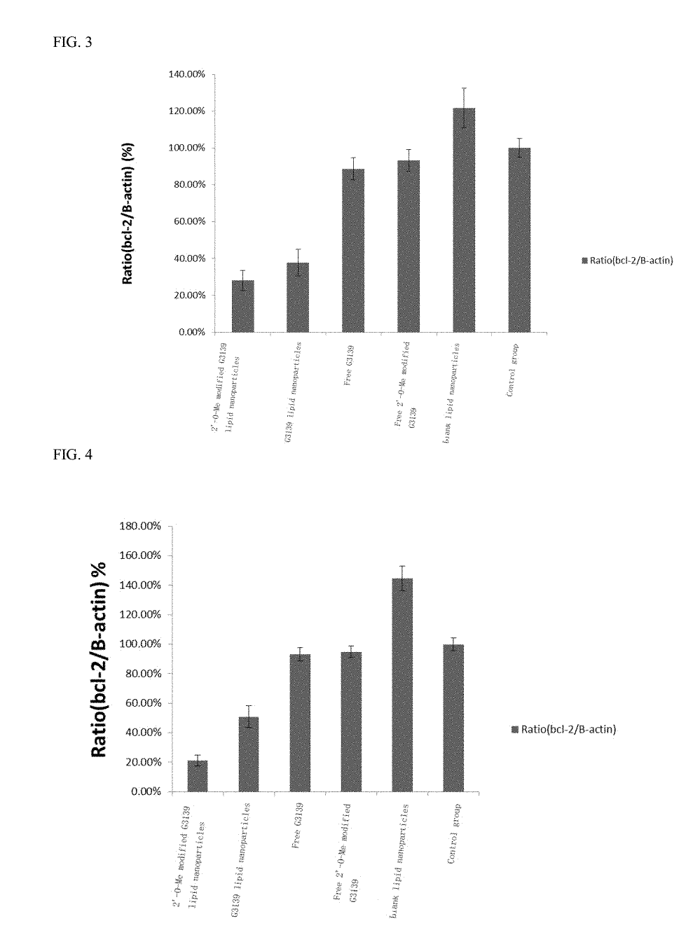

The test results are as shown in the following table and FIG. 3:

| | Ratio | Standard |
|---|---|---|
| | (bcl-2/β-actin) | deviation |
| 2′-O-Me modified G3139 lipid nanoparticles | 28.13% | 5.46% |
| G3139 lipid nanoparticles | 37.65% | 7.27% |
| free G3139 | 88.75% | 6.11% |
| free 2′-O-Me modified G3139 | 93.29% | 5.93% |
| blank lipid nanoparticles | 121.67% | 10.81% |
| control group | 100% | 5.09% |

From the above table and FIG. 3, the 2′-O-Me modified lipid nanoparticles have a 71.87% gene-level down-regulation, as compared with other experimental groups, the bcl-2 level has a large degree of down-regulation, while the free drug group (3,4) cannot enter the cells due to the lack of a drug delivery system, but only has a weak downregulation (11.25% and 6.71%), and blank nanoparticles cannot play a role of down-regulation because there is no drug.

3) LNCaP Prostate Cancer Cells

The test results are as shown in the following table and FIG. 4:

| | Ratio | Standard |
|---|---|---|
| | (bcl-2/β-actin) | deviation |
| 2′-O-Me modified G3139 lipid nanoparticles | 21.22% | 3.68% |
| G3139 lipid nanoparticles | 50.92% | 7.27% |
| free G3139 | 93.15% | 4.46% |
| free 2′-O-Me modified G3139 | 94.89% | 3.95% |
| blank lipid nanoparticles | 144.73% | 8.33% |
| control group | 100% | 4.31% |

100%4.31% From the above table and FIG. 4, the 2′-O-Me modified lipid nanoparticles have a 78.78% gene-level down-regulation, as compared with other experimental groups, the bcl-2 level has a large degree of down-regulation, while the free drug group (3,4) cannot enter the cells due to the lack of a drug delivery system, but only has a weak downregulation (6.85% and 5.11%), and blank nanoparticles cannot play a role of down-regulation because there is no drug.

3. Pharmacodynamics of ectopically inoculating KB cells into tumor cell models in mice and survival curve test

(1) 5*10⁶ KB cancer cells will be inoculated into the right back of nude mice and allowed to grow for 1-2 weeks to a visible tumor of an average 150 mm³.

(2) When the tumors reach the expected size, each group of tumor-bearing mice will be given different types of drugs through the caudal vein, namely 2′-O-Me modified G3139 lipid nanoparticles, G3139 lipid nanoparticles, free G3139, free 2′-O-Me modified G3139, paclitaxel injection, and normal saline. Drugs are given once every three days for three weeks. The treated groups are divided into 6 groups with 10 tumor-bearing mice in each group.

(3) The size including length and width of the tumor will be measured by the Vernier caliper, and volume of the tumor is given by the following formula:

Tumor volume=(π/6)*length (mm)*(width)²

(4) When the actual tumor size reaches 1500 mm³, the mice will be removed from the treated group and euthanized through carbon dioxide asphyxiation.

The volume of tumor in each experimental group after the end of the treatment cycle is an indication of antitumor activity.

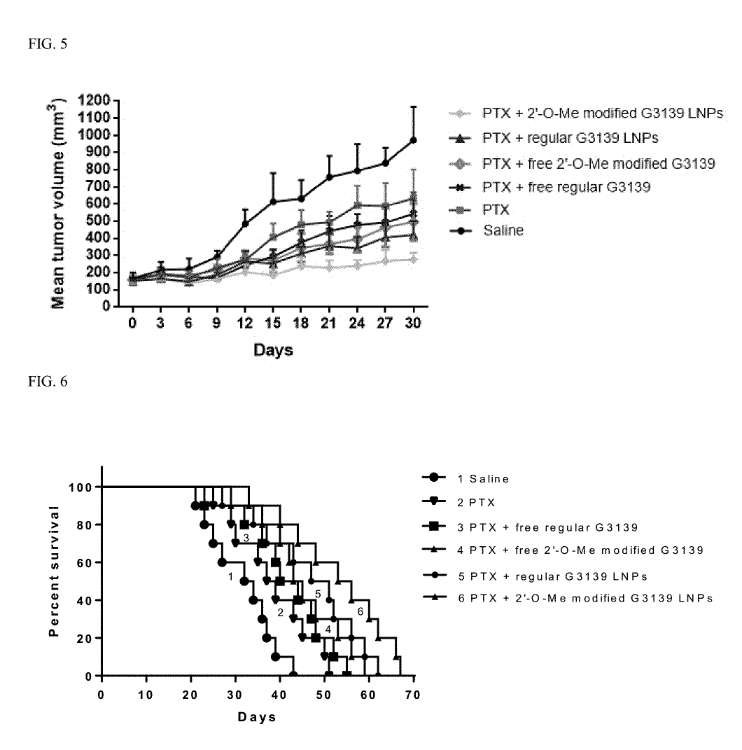

FIG. 5 and FIG. 6 are graphs of the tumor inhibition rate test and the survival test.

From FIG. 5 and FIG. 6, and from the perspective of the pharmacodynamics of xenografts of KB cells in nude mice and the survival curve, we can see that the 2′-O-Me modified G3139 lipid nanoparticles are combined with paclitaxel to exert fairly good tumor inhibiting effect and long survival time of tumor-bearing mice, which indicates that the lipid nanoparticles do not produce significant toxic side effects while improving the efficacy of the drug.

1. A lipid nanoparticle for antisense oligonucleotides inhibiting bcl-2, wherein the lipid nanoparticle is prepared by coating an antisense oligonucleotide with a membrane material, and a nucleic acid sequence is 5′-TCT CCC AGC GTG CGC CAT-3′ (SEQ ID NO: 1), or 5′ UCU CCC AGC GTG CGC CAU 3′ (SEQ ID NO: 2); and
the membrane material comprises a cationic lipid, a neutral phospholipid, cholesterol, Tween, a polyethylene glycol derivative in a molar ratio of (25-35):(40-50):(15-25):(1-5):(1-5).

2. The lipid nanoparticle for antisense oligonucleotides inhibiting bcl-2 according to claim 1, wherein the cationic lipid includes: DOTAP, DOTMA, DDAB, and DODMA;
the neutral phospholipid includes: egg PC, DOPC, DSPC, DPPC, and DMPC;
the Tween includes: Tween-20, Tween-40, Tween-60, and Tween-80; and
the polyethylene glycol derivative includes: mPEG-DPPE, mPEG-DMPE, mPEG-DSPE, TPGS, and mPEG-cholesterol.

3. The lipid nanoparticle for antisense oligonucleotides inhibiting bcl-2 according to claim 1, wherein the PEG in the polyethylene glycol derivative includes monomethoxy polyethylene glycol having a molecular weight of from 550 to 5,000.

4. The lipid nanoparticle for antisense oligonucleotides inhibiting bcl-2 according to claim 3, wherein the polyethylene glycol derivative is a substance formed by linking a hydrophilic polyethylene glycol chain or a methoxy polyethylene glycol chain to a hydrophobic structure that can be embedded into a phospholipid bilayer; the hydrophobic structure includes cholesterol, vitamin E, dipalmitoylphosphatidylethanolamine, dimyristoylphosphatidylethanolamine, dimyristoylglycerol or structural analogs.

5. The lipid nanoparticle for antisense oligonucleotides inhibiting bcl-2 according to claim 2, wherein the Tween is Tween-80.

6. The lipid nanoparticle for antisense oligonucleotides inhibiting bcl-2 according to claim 1, wherein a modification to the antisense oligonucleotide 5′ UCU CCC AGC GTG CGC CAU 3′ (SEQ ID NO: 2) is a 2′-O-Me modification to 3 nucleotides at both ends thereof, and a phosphorothioate modification to an entire chain; or phosphorothioate modification to an entire chain of antisense oligonucleotide 5′-TCT CCC AGC GTG CGC CAT-3′ (SEQ ID NO: 1).

7. A method for preparing a lipid nanoparticle for antisense oligonucleotides inhibiting bcl-2, wherein the method for preparing the lipid nanoparticle is an ethanol stepwise dilution method comprising the following specific steps:
(1) dissolving a cationic lipid, a neutral phospholipid, cholesterol, Tween, and a polyethylene glycol derivative in 80% ethanol in a certain molar ratio to obtain a mixed ethanol solution; and dissolving the modified antisense oligonucleotides in a PBS buffer to obtain a PBS solution of antisense oligonucleotides;
(2) mixing the resulting mixed ethanol solution and the PBS solution of antisense oligonucleotides in equal volumes to obtain a 40% final ethanol concentration mixed solution;
(3) further diluting the 40% final ethanol concentration mixed solution obtained in step (2) with the PBS solution in an equal volume; repeatedly diluting the final ethanol concentration mixed solution with the PBS solution in an equal volume until a preparation mixed solution with a final ethanol concentration of less than 5% is obtained;
(4) adding a high-salinity solution to the preparation mixed solution obtained in step (3) to obtain a mixed solution containing high-concentration of salt;
(5) removing ethanol and free uncoated oligonucleotide from the mixed solution obtained in step (4) with an ultrafiltration or dialysis device;
(6) filtering and sterilizing a product obtained in step (5) through a filter membrane or a filter element with a pore size of less than or equal to 0.22 μm to obtain the lipid nanoparticle.

8. The method for preparing a lipid nanoparticle according to claim 7, wherein the PBS buffer does not contain DNase and RNase, the PBS buffer in the step (1) has a specification of 1×pH=7, and the PBS buffer in the step (3) has a specification of 1×pH=7.4.

9. The method for preparing a lipid nanoparticle according to claim 7, wherein the high-salinity solution is a NaCl solution, and the concentration of NaCl in the mixed solution in the step (4) is 0.1-1 M.

10. The method for preparing a lipid nanoparticle according to claim 9, wherein the concentration of the mixed solution is 0.3-0.4 M.

11. The method for preparing a lipid nanoparticle according to claim 7, wherein the ultrafiltration or dialysis device has a molecular weight cutoff of 10,000-100,000 daltons.

12. The lipid nanoparticle for antisense oligonucleotides inhibiting bcl-2 according to claim 2, wherein the membrane material comprises DOTAP, Egg PC, Cholesterol, Tween 80, and TPGS in a molar ratio of 25:45:20:5:5.

13. The lipid nanoparticle for antisense oligonucleotides inhibiting bcl-2 according to claim 2, wherein the membrane material comprises DOTMA, Egg PC, Cholesterol, Tween 80, and TPGS in a molar ratio of 30:50:20:5:5.

14. The lipid nanoparticle for antisense oligonucleotides inhibiting bcl-2 according to claim 2, wherein the membrane material comprises DOTAP, DSPC, Cholesterol, Tween 80, and TPGS in a molar ratio of 30:50:20:5:5.

15. The lipid nanoparticle for antisense oligonucleotides inhibiting bcl-2 according to claim 2, wherein the membrane material comprises DOTAP, Egg PC, Cholesterol, Tween 80, and mPEG2000-DPPE in a molar ratio of 30:45:20:5:5.
